(19) United States
(12) Patent Application Publication
(10) Pub. No.: US 2003/0086978 A1
(43) Pub. Date: May 8, 2003

(54) Photochromic nanocapsule and preparation method thereof

(75) Inventors: Eun Kyoung Kim, Yusung-ku (KR); Mijeong Han, Yusung-ku (KR)

Correspondence Address: Finnegan, Henderson, Farabow,, Garrett & Dunner, L.L.P., 1300 I Street, N.W., Washington, DC, 20005-3315, US

(73) Assignee: KOREA RESEARCH INSTITUTE OF CHEMICAL TECHNOLOGY

(21) Appl. No.: 10/171,590

(22) Filed: Jun. 17, 2002

(30) Foreign Application Priority Data
Sep. 18, 2001 (KR) 2001-57442

(51) Int. Cl. A61K 9/50; B32B 23/02; B32B 21/02; B32B 19/00; B32B 17/02; B32B 15/02; B32B 9/04; B32B 9/02; B32B 9/00; B32B 5/16; B32B 27/02; G02B 5/23

(52) U.S. Cl. 424/501; 252/586; 428/402.24

(57) ABSTRACT

The present invention relates to a photochromic nanocapsule and its preparation method thereof, and more particularly, to a core/shell type photochromic nanocapsule comprising a photochromic diarylethene compound encompassed by polymers and its preparation method thereof, wherein a diarylethene compound, a monomer or a mixture of monomers, an emulsifier and a initiator are emulsified followed by the polymerization at a predetermined temperature thus resulting in a photochromic nanocapsule which is not only reduced in size from μm of the conventional ones to nm but also has an excellent thermal stability, transparency, and an excellent photochromic property thereby enabling to be used as optical materials such as an optical recording material, an optical switch, and the like.

### FIELD OF THE INVENTION

The present invention relates to a photochromic nanocapsule and its preparation method thereof, and more particularly, to a core/shell type photochromic nanocapsule comprising a photochromic diarylethene compound encompassed by polymers and its preparation method thereof, wherein a diarylethene compound, monomer(s), an emulsifier and a polymerization initiator are emulsified followed by the polymerization at a predetermined temperature thus resulting in a photochromic nanocapsule which is not only reduced in size from μm of the conventional ones to nm but also has excellent heat stability and excellent photochromic property thereby enabling to be used as optical materials such as an optical recording material, an optical switch, and the like.

### BACKGROUND OF THE INVENTION

The colors of the photochromic compounds are reversibly changed when they are exposed to light. There have been known various photochromic compounds with various structures such as spirobenzopyran, azobenzene, formazan, fulgide, naphthopyran, and the like. Of them, diarylethene photochromic compounds, which were first synthesized in 1985, are shown to be thermally stable by not changing their colors by heat but change colors only when exposed to photo irradiation [Japan Kokai Tokkyo Koho; JP 86263936 A2; Bell, S. I.; Parvez, M.; Weinreb, S. M. J. Org. Chem., 1991, 49, 373]. Since then, studies have been focused on developing methods of synthesizing various kinds of derivatives and many lines of researches have been published on reversible optical discs, rewritable optical discs, optical switches for optical integrated elements, organic photosensitizers and the like [Japan Kokai Tokkyo Koho; JP 9761647 A2, JP 9780681 A2; Takeshita, M; Uchida, K; Irie, M. Chem. Commun., 1996, 1807˜1808].

Diarylethene compounds, being highly stable in a dark state as well as efficiently photochromic, can be applied in manufacturing optical recording films by a solution coating method wherein said diarylethene compounds are dissolved along with a polymer resin [Bell, S. I.; Parvez, M.; Weinreb, S. M. J. Org. Chem., 1991, 49, 373]. However, introduction of a diarylethene compound into a polymer resin results in difficulty in forming a homogeneous film due to insufficient compatibility with a polymer resin and also results in low photochromic efficiency due to aggregation between photochromic molecules. In addition, if high content of diarylethene compound is introduced into polymer resin, the resulting film becomes opaque because of a phase separation between diarylethene photochromic compounds and the polymer. Therefore, there has been raised a problem with regard to reliance and storage stability of the optical recording.

Korea Unexamined Patent Publication No. 95-9349 discloses a method of manufacturing a photochromic capsule using a composition comprising 0.1-1 wt % of spiropyran-based compound, oil and gelatin and the like, however, the spiropyran-based compound was shown poor at heat stability as well as photo stability thus resulting in poor stability of the resulting manufactured photochromic compounds. Moreover, thus manufactured photochromic compounds had poor translucency due to their sizes being a few micrometers in diameter and poor photochromic property.

### SUMMARY OF THE INVENTION

To solve the above problems, the inventors of the present invention developed a method, wherein photochromic material is encompassed with polymers as a way to prevent the aggregation among photochromic materials, and studied a way to introduce photochromic material into a capsule of core/shell type. In particular, the inventors implemented intensive studies to find a way to reduce the size of a photochromic capsule less than 200 nm, i.e., smaller than the wavelength of visible light, in order to manufacture a transparent film with excellent photochromic property. Then, the inventors prepared a composition comprising a photochromic compound, a monomer and an emulsifier in such a manner that monomers can encompass the photochromic compound, and immediately polymerized them to obtain a composition containing capsules ranging 15-200 nm in size. Thus obtained composition was then used in manufacturing photochromic nanocapsule powder having excellent photochromic property and the film containing the same.

Therefore, the object of the present invention is to provide a core/shell type nanocapsule by using a composition comprising diarylethene compound having excellent photochromic property, an emulsifier and an initiator and also provide a photochromic film therefrom.

### BRIEF DESCRIPTION OF THE DRAWINGS

FIG. 1 is a graph that shows the change in spectroscopic absorbance spectrum after irradiation of 365 nm short wavelength to the photochromic nanocapsule manufactured in Example 11.

FIG. 2 shows the microscopic view of the nanocapsule manufactured in Example 1 observed by using a transmission electron microscope.

### DETAILED DESCRIPTION OF THE INVENTION

The present invention relates to a photochromic nanocapsule composition which comprises 0.1-40 parts by wt of a diarylethylene compound, 15-99 parts by wt of a monomer or a mixture of monomers, 0.01-10 parts by wt of an emulsifier, 0.1-10 parts by wt of an initiator for polymerization and 50-90 parts by wt of water, and is characterized in that the polymerized product of said monomer(s) is a capsule that encompasses said diarylethylene compound, wherein said diarylethylene compound is represented by the following formula 1,

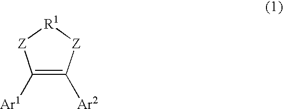

wherein R¹ is a bonding line, nothing (no chemical bonding), an alkylene group having carbon atoms of 1-3, or an alkylene group having carbon atoms of 1-3 substituted with fluoride; Ar¹ and Ar² are represented by the following formula 2 or 3, respectively; and Z is CH₂, CF₂, CN, or CO, respectively;

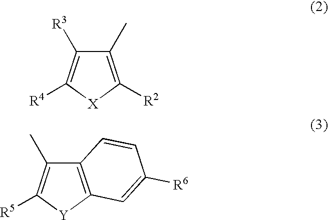

wherein R² and R⁵ are independently an alkyl group having carbon atoms of 1-3 which is either substituted with a fluoride atom or a hydrogen atom, respectively; R³ is the same as R² or R⁵, or a hydrogen atom or a fluoride atom; R⁴ and R⁶ are independently the same as R³, or a phenyl isoxazole group, a hydroxymethyl isoxazole group, an alkyleneoxy alkyl ester group, an aldehyde group, a carboxylic acid group, —[CH═CH]1—[C(═O)]ₘ[CH2]ₙ—[CH═CH]₀—C(R⁷)ₚ(R⁸)_{q} or —C≡C—R⁹; R⁷, R⁸ and R⁹ are independently a hydrogen atom, an alkyl group having carbon atoms of 1-22 or a phenyl group; 1, n, o are independently a whole number ranging from 0 to 10; m is 0 or 1; p and q are independently a whole number of from 0 to 3 wherein p+q−3; and X and Y are independently an oxygen, a nitrogen or a sulfur atom.

The examples of diarylethene compounds of the above formula I are 1,2-bis(2-methylbenzo[b]thiophene-3-yl)hexafluorocyclopentene(BTF6, Structure 1),

1, [6′-(methacryoloxyethyloxycarbonyl)-2′-methylbenzo[b]thiophene-3′-yl]-2-(2″-methylbenzo[b]thiophene-3″-yl)hexafluorocyclopentene(MMBTF, Structure 2), cis-1,2-bis(2-methylbenzothiophene-3-yl)-1,2-dicyanoethene(BTCN, Structure 3), cis-1,2-dicyano-1,2-bis(2,4,5-trimethyl-3-thienyl)ethane(MTCN, Structure 4) and the like and they are shown in the following structures of 1-15.

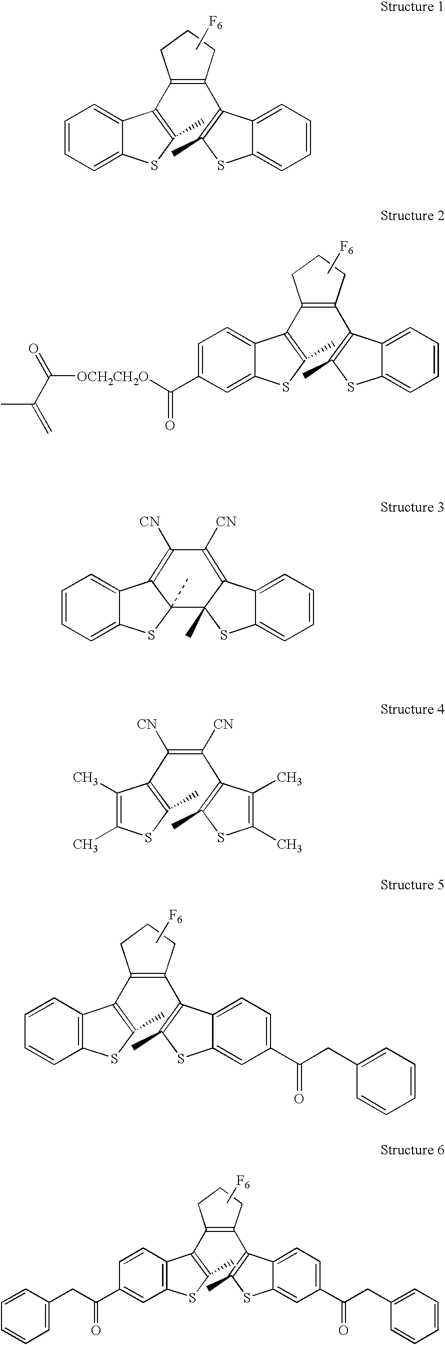

The diarylethene compounds shown in the above formula 1 and the structures of 1-15 have excellent photochromic property and it is preferred to use 0.1-40 parts by wt of at least one compound selected from the above group. If the amount of diarylethene compounds used is off the above range, nanocapsules cannot be manufactured due to the occurrence of aggregation among the diarylethene compounds.

Examples of the monomer(s) are one or a mixture of more than two compounds having an unsaturated group selected from the group consisting of styrene, alkylacrylate, polyalkyleneglycol acrylate, acrylic acid and vinyl cabazole, which are either substituted or non-substituted.

Examples of the emulsifiers are sodium dodecylsulfate(SDS) or sodium laurylsulfate and the preferred amount of use is 0.01-10 parts by wt.

Examples of the initiators for the polymerization are N,N′-azobisisobutyronitrile, potassium persulfate and azo-polyethylene glycol and the like and the preferred amount of use is 0.1-10 parts by wt. The above azo-polyethylene glycol can be represented by the following formula 4 and it can be synthesized by using N,N′-azobisisobutyronitrile and polyethylene glycol (M.W.=200-5000).

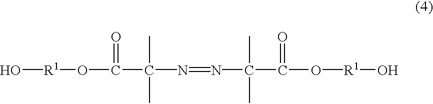

In the above formula 4, R¹ represents ethyl, butyl, dioxaoctyl, triethylene oxide, and polyethylene oxide.

Further, a compound selected from the group consisting of spirobenzopyran, formazan, naphthopyran, fulgide, azobenzene, disperse red, disperse orange, spirooxazine, phthalocyanine, pigments, dyes, and pharmaceutical drugs can be added in addition to the above diarylethene compound, wherein said compound can be added 0.05-15 wt % of the total composition for a capsule. These compounds can be either purchased from Aldrich Co. or Tokyo Kasei Co. and the like or synthesized by using a method known to a skilled person of the art. Still further, an antioxidant, a thickener, an organic solvent, a surfactant, a UV inhibitor and the like can be used additionally.

The manufacturing steps of the photochromic nanocapsules of the present invention are described as follows.

First, a diarylethene compound, a monomer or a mixture of monomers, an initiator and water are mixed and stirred in an ice-bath. The resulting mixture is then vehemently vibrated for emulcification by using a mechanical stirrer, a homogenizer or a sonicator. The above initiator can be used in the polymerization by adding it before or after the emulsification, and it can be added additionally when manufacturing films using the capsule composition obtained from the polymerization.

Second, a polymerization is conducted by heating at 60-70° C. Monomers can be polymerized at this temperature to form a polymer and manufacture a core/shell type capsule containing diarylethene compound. Thus obtained nanocapsule composition is coated on silicon wafers or glass plate by means of a coating solution method followed by drying at room temperature thus finally manufacturing a photochromic film. The photochromic film showed excellent absorbance at wavelength of 300-800 nm when exposed to light or UV irradiation.

Further, the above photochromic nanocapsule composition can be applied to a variety of fields for the purpose of imparting photochromic property such as optical recording media, photochromic windows, display elements, plastic mirrors, photochromic filters, photosensing drums, recording elements, solar batteries, lenses, fibers, photochromic recording and image parts, or pharmaceutical drugs.

Hereunder is given a detailed description of the present invention using the following Examples, however, it should not be construed as limiting the scope of the present invention.

The materials used in the following Examples such as a photochromic compound, an initiator and a solvent were either synthesized by using a method known to a skilled person of the art or purchased from Aldrich Co., Tokyo Kasei Co., etc.

The properties were evaluated by the following test methods.

[Test Method]

(1) Thickness: Measured by using α-Step 200.

(2) Photochromicity: Measured by using a UV/Vis spectroscope.

(3) Transmission: Measured by using 2 mm thick test samples via UV/Vis spectrum at 400-800 nm and the transmission values shown were taken from those at 700 nm.

(4) Measurement of Diameter of Photochromic nanocapsule: Ultrathin films were prepared to measure the shape and the size of photochromic capsules. That is, an emulsion solution diluted to about 0.01 wt % was dropped onto 200-mesh carbon-coated copper grid and then dried. The shape and the size of thus prepared photochromic capsule samples were observed by transmission electron microscopy(TEM).

(5) Molecular Weight: Polymers were dissolved again in THF after polymerization and their molecular weights were analyzed by using gel permeation chromatography(GPC).

(6) Thermal stability: Sample was analyzed by Thermogravimetric analyses (TGA) using Du Pont 951 thermogravimetric analyzer. The sample weight was 3-10 mg.

### Preparation Example 1

### Synthesis of Azopolyethylene Glycol(PEGA)

A mixture consisting of 1.64 g of N,N′-azobisisobutyronitrile(AIBN), 10 g of polyethylene glycol (MW=300) and 20 mL of benzene was saturated with HCl gas at 5° C. and allowed to react for 12 hr. Upon completion of the reaction, the top fraction of the mixture consisting of colorless benzene layer was removed and the bottom fraction was dropped into a mixture containing 15 mL of water and 20 g of ice and organic fraction was collected. Water fraction was removed while extracting the organic fraction with chloroform. Thus collected organic fraction was neutralized with saturated solution of sodium carbonate and then water was removed by using magnesium sulfate. Chloroform, a solvent, was evaporated under reduced pressure and dried completely in a vacuum oven.

### EXAMPLE 1

### Manufacture of Capsules Using 1,2-bis(2-methylbenzo[b]thiophene-3-yl)hexafluorocyclopentene(BTF6)

1.5 g of 1,2-bis(2-methylbenzo[b]thiophene-3-yl)hexafluorocyclopentene(BTF6) was dissolved in 4.5 g of purified styrene, added with 99 mg of N,N′-azobisisobutyronitrile(AIBN) and the mixture was stirred for 10 min. In a separate container, 250 mg of SDS was dissolved in 30 g of water, added with the above mixture, stirred for 30 min and finally obtained an emulsion solution. The emulsion solution was treated with ultrasonification for 120 sec and all the above processes were performed in an ice bath to prevent polymerization of styrene. The reaction mixture was slowly heated to 70° C. and polymerized for 18 hr at 70° C. while stirring at 400 rpm. Upon completion of the reaction, the temperature was slowly lowered to room temperature and manufactured nanocapsules with 70 nm in diameter wherein 1,2-bis(2-methylbenzo[b]thiophene-3-yl)hexafluorocyclopentene(BTF6) is encompassed with polystyrene. The transmittance of thus manufactured nanocapsule solution was higher than 90% at 50 μm in diameter and the color of the solution turned from colorless to red when exposed to a UV irradiation. FIG.2 shows a picture of thus obtained nanocapsules taken by using a transmission electron microscope and a bar code is equivalent to 100 nm. The photochromic capsule was stable up to 400° C. without loss of weight by decomposition, as determined by TGA.

### EXAMPLE 2

### Manufacture of Capsules Using 1,[6′-(methacryloxyethyloxycarbonyl)-2′-methylbenzo[b]thiophene-3′-yl]-2-(2″-methylbenzo[b]thiophene-3″-yl)hexafluorocyclopentene(MMBTF)

60 mg of 1,[6′-(methacryloxyethyloxycarbonyl)-2′-methylbenzo[b]thiophene-3′-yl]-2-(2″-methylbenzo[b]thiophene-3″-yl)hexafluorocyclopentene(MMBTF) was dissolved in 2.32 g of purified styrene, added with 52 mg of N,N′-azobisisobutyronitrile(AIBN) and the mixture was stirred for 10 min. In a separate container, 12 mg of SDS was dissolved in 12 g of water, added with the above mixture, and mixed for 30 min by using an ultrasonic homogenizer. All the above processes were performed in an ice bath to prevent polymerization of styrene. The reaction mixture was slowly heated to 70° C. and polymerized for 18 hr at 70° C. while stirring at 400 rpm. Upon completion of the reaction, the temperature was slowly lowered to room temperature and manufactured photochromic nanocapsules with 50-150 nm in diameter. The transmittance of thus manufactured nanocapsule solution was higher than 90% at 50 μm in diameter and the color of the solution turned from colorless to red when exposed to a UV irradiation.

### EXAMPLE 3

### Manufacture of Capsules Using cis-1,2-dicyano-1,2-bis(2,4,5-trimethyl-3-thienyl)ethane(MTCN)

60 mg of cis-1,2-dicyano-1,2-bis(2,4,5-trimethyl-3-thienyl)ethane(MTCN) was dissolved in 2.32 g of purified styrene, added with 52 mg of N,N′-azobisisobutyronitrile(AIBN) and the mixture was stirred for 10 min. In a separate container, 12 mg of SDS was dissolved in 12 g of water, added with the above mixture, and mixed for 30 min by using an ultrasonic homogenizer. All the above processes were performed in an ice bath to prevent polymerization of styrene. Thus obtained emulsion solution was slowly heated to 70° C. and was polymerized for 12 hr at 70° C. while stirring at 400 rpm. Upon completion of the reaction, the temperature was slowly lowered to room temperature and manufactured photochromic nanocapsules with 50 nm in diameter. The transmittance of thus manufactured nanocapsule solution was higher than 90% at 50 μm in diameter and the color of the solution turned from colorless to red when exposed to a UV irradiation.

### EXAMPLE 4

### Manufacture of Capsules Using cis-1,2-bis(2-methylbenzothiophene-3-yl)-1,2-dicyanoethene(BTCN)

30 mg of cis-1,2-bis(2-methylbenzothiophene-3-yl)-1,2-dicyanoethene(BTCN) was dissolved in 1.16 g of purified styrene, added with 26 mg of N,N′-azobisisobutyronitrile(AIBN) and the mixture was stirred for 10 min. In a separate container, 6 mg of SDS was dissolved in 6 g of water, added with the above mixture, and mixed for 30 min by using an ultrasonic homogenizer. All the above processes were performed in an ice bath to prevent polymerization of styrene. Thus obtained emulsion solution was slowly heated to 75° C. and polymerized for 15 hr at 75° C. while stirring at 400 rpm. Upon completion of the reaction, the temperature was slowly lowered to room temperature and manufactured photochromic nanocapsules with 60-70 nm in diameter.

### EXAMPLE 5

### Manufacture of Capsules Using 1,2-bis(2-methylbenzo[b]thiophene-3-yl)hexafluorocyclopentene(BTF6)

60 mg of 1,2-bis(2-methylbenzo[b]thiophene-3-yl)hexafluorocyclopentene(BTF6) was dissolved in 2.32 g of purified styrene. In a separate container, 12 mg of SDS was dissolved in 12 g of water, added with the above mixture, and mixed for 30 min by using an ultrasonic homogenizer. All the above processes were performed in an ice bath to prevent polymerization of styrene. Thus obtained emulsion solution was added with 52 mg of azo-polyethylene glycol (PEGA200). The reaction mixture was slowly heated to 60° C. and was polymerized for 12 hr at 60° C. while stirring at 400 rpm. Upon completion of the reaction, the temperature was slowly lowered to room temperature and manufactured photochromic nanocapsules with 15-80 nm in diameter.

### EXAMPLES 6-10

Experiments were performed the same as in Example 1 except that diarylethene compounds, monomers, emulsifiers, and initiators were modified as shown in the following Table 1. The amount of water used was 12 g, respectively.

TABLE 1

| | | | | | | Ave. |
|---|---|---|---|---|---|---|
| | | | | | | Diameter |
| | | | | | | of |
| Classifi- | | Diarylethene | | Emulsifier | Initiator | Capsule |
| cation | | compound | Monomer(g) | (g) | (g) | (nm) |
| Ex. | 6 | BTF6¹⁾(0.06) | Styrene(2.3) | SDS(0.012) | AIBN³⁾ | 70 |
| | | | | | (0.05) | |
| | 7* | BTF6 (0.06) | Styrene(2.3) | SDS(0.012) | KPS⁴⁾ | 60 |
| | | | | | (0.052) | |
| | 8 | Structure 3 | Styrene(2.3) + | SDS(0.011) | AIBN³⁾ | 75 |
| | | (0.06) | Methylacrylate | | (0.05) | |
| | | | (2.2) | | | |
| | 9* | BTF6 (0.06) | Styrene(2.3) | SDS(0.012) | PEGA⁵⁾ | 50 |
| | | | + Vinyl | | (0.06) | |
| | | | carbazole(0.5) | | | |
| | 10 | Structure 2 | Styrene(2.1) | SDS(0.011) | PEGA | 45 |
| | | (0.1) + | | | (0.04) | |
| | | SP²⁾(0.02) | | | | |

¹⁾BTF6: 1,2-bis(2-methylbenzo[b]thiophene-3-yl)hexafluorocyclopentene
²⁾SP: 6-nitro spirobenzopyran
³⁾AIBN: N,N′-azobisisobutyronitrile
⁴⁾KPS: potassium sulfate
⁵⁾PEGA: azo-polyethylene glycol (Preparation Example 1)
*In Examples 7 and 9, an initiator is added for polymerization after an emulsion is obtained.

### Comparative Example 1

### Manufacture of Photochromic Capsules and Their Films According to a Known Method

Photochromic capsules were manufactured by using the known method disclosed in Korea Patent No. 127771. Sixty mg of the compound of structure 2 was added into 2.7 g of oil and maintained at 60-70° C. by heating (solution A). 1.8 g of gelatin was dissolved in 10 g of water by heating and maintained at 70-75° C. (solution B).

Thus manufactured solution B was stirred at 3000 rpm while adding the solution A at the rate of 5 mL/s. To water of about 50° C. was added 0.36 g of m-xylenediamine, an amine-based hardening agent. After stirring the above emulsion for 10 min, was added 0.6 g of epoxy resin, stirred again for 5 min and then dropped the hardening solution. Water evaporated during the reaction was replenished by adding water of about 70-75° C. to maintain the initial volume of water. After completion of the reaction, a little amount of cold water is added to make the total volume of 12 mL and then solidified capsule type photochromic composition can be obtained. Thus manufactured capsules were larger than 300 nm in size and the permeability of the emulsion solution was less than 10% thus resulting in large aggregation of capsules. The effect of color comparison was not able to estimate due to the low permeability. The photochromic capsule was decomposed at 120° C. with 5% loss of weight by decomposition, as determined by TGA.

### EXAMPLE 11

### Manufacture of Films Using Photochromic Nanocapsules

The photochromic capsules manufactured in the above Example 1 was filtered through an injection filter of 0.45 μm in size, coated on top of a glass plate by using a spin coater, and dried in a vacuum oven kept at room temperature for 12 hr to finally obtain a transparent film with excellent adhesiveness and high transparency (transmission=90%). The color of thus manufactured film turns into red when exposed to light of greater than 30 nm and this red color is maintained if the film is stored in a dark room where light is completely blocked.

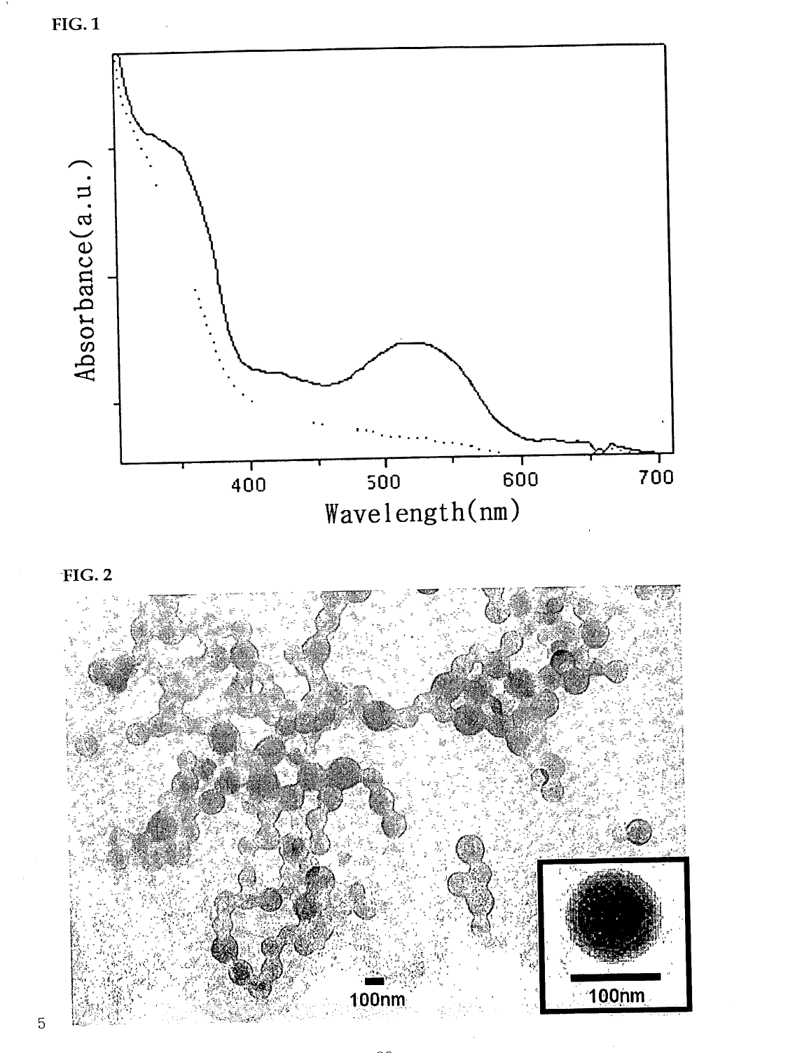

FIG. 1 shows the change in absorption spectra of capsule films irradiated by short wavelength of 365 nm, respectively, wherein BTF6 is encompassed with polystyrenes (dotted line: before light exposure, solid line: 5 min after light exposure).

### EXAMPLES 12-15

### Manufacture of Films Using Photochromic Nanocapsules

Photochromic films were manufactured by changing the compositions and manufacturing conditions in Example 11 as shown in the following Table 2.

TABLE 2

| Classifi- | | Nanocapsule | Additive | Film Manufacturing | Photochromic | Transmis- |
|---|---|---|---|---|---|---|
| cation | | Composition | (wt %) | Conditions | color change | sion (%) |
| Example | 12 | Example 2 | — | Dry after spin coating | Colorless-red | 90 |
| | 13 | Example 3 | PEGA*(0.1) | Dry after spin coating, | Colorless-red | 95 |
| | | | | Placing at vacuum | | |
| | | | | oven at 80° C. | | |
| | 14 | Example 5 | — | Dry after spin coating, | Colorless-red | 93 |
| | | | | Placing at vacuum | | |
| | | | | oven at 60° C. | | |
| | 15 | Example 9 | Irgacure | Dry after spin coating, | Colorless-red | 85 |
| | | | 184**(0.1) | Exposure to UV for 3 | | |
| | | | | min | | |
| | 16 | Example 10 | — | Dry after spin coating, | Colorless-pur- | 87 |
| | | | | Placing at vacuum | ple | |
| | | | | oven at 60° C. | | |

*PEGA: azo-polyethyleneglycol (preparation example 1)
**Irgacure 184: Product of Ciba Geigy Co., Ltd.

As described in the above, the present invention relates to manufacturing core/shell type photochromic capsules via emulsion polymerization of diarylethene compounds and monomers and subsequent manufacturing of photochromic films with excellent heat stability and photochromic efficiency. The method of manufacturing photochromic nanocapsules employed in the present invention can be applied to nanocapsulization of photochromic agents such as spirobenzopyran, formazan, naphthopyran, fulgide, azobenzene, disperse red, disperse orange, spirooxazine, phthalocyanine and the like. Further, this method can be also effectively used in manufacturing parts for photochromic recording, image parts, drugs, lenses and the like by adding pigments and pharmaceutical agents.

What is claimed is:

1. A photochromic nanocapsule composition which comprises 0.1-40 parts by wt of a diarylethylene compound, 15-99 parts by wt of monomer(s), 0.01-10 parts by wt of an emulsifier, 0.1-10 parts by wt of an initiator for polymerization and 50-90 parts by wt of water, and is characterized in that the polymerized product of said monomer(s) is a capsule that encapsulate said diarylethylene compound.

2. The photochromic nanocapsule composition according to claim 1, wherein the size of said capsule is 5-200 nm in diameter.

3. The photochromic nanocapsule composition according to claim 1, wherein said diarylethylene compound is represented by the following formula 1, 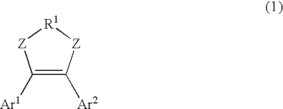
wherein R¹ is a bonding line, nothing (no chemical bonding), an alkylene group having carbon atoms of 1-3, or an alkylene group having carbon atoms of 1-3 substituted with fluoride; Ar¹ and Ar² are represented by the following formula 2 or 3, respectively; and Z is CH₂, CF₂, CN, or CO, respectively; 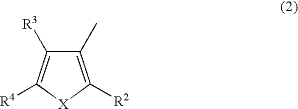 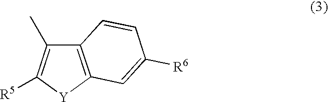
wherein R² and R⁵ are independently an alkyl group having carbon atoms of 1-3 which is either substituted with a fluoride atom or a hydrogen atom, respectively; R³ is the same as R² or R⁵, or a hydrogen atom or a fluoride atom; R⁴ and R⁶ are independently the same as R³, or a phenyl isoxazole group, a hydroxymethyl isoxazole group, an alkyleneoxy alkyl ester group, an aldehyde group, a carboxylic acid group, —[CH═CH]ₗ—[C(═O)]ₘ[CH2]ₙ—[CH═CH]ₒ—C(R⁷)ₚ(R⁸)_{q} or —C≡C—R⁹; R⁷, R⁸ and R⁹ are independently a hydrogen atom, an alkyl group having carbon atoms of 1-22 or a phenyl group; l, n, o are independently a whole number ranging from 0 to 10; m is 0 or 1; p and q are independently a whole number of from 0 to 3 wherein p+q=3; and X and Y are independently an oxygen, a nitrogen or a sulfur atom.

4. The photochromic nanocapsule composition according to claim 1, wherein said monomer(s) is one or a mixture of more than two selected from the group consisting of styrene, alkylacrylate, polyalkyleneglycol acrylate, acrylic acid and vinyl cabazole, which are either substituted or non-substituted.

5. The photochromic nanocapsule composition according to claim 1, wherein a compound selected from the group consisting of spirobenzopyran, formazan, naphthopyran, fulgide, azobenzene, disperse red, disperse orange, spirooxazine, phthalocyanine, pigments, dyes, and pharmaceutical drugs, is added in addition to said diarylethene compound.

6. A method of manufacturing photochromic nanocapsule composition comprising 0.1-40 parts by wt of a diarylethylene compound, 15-99 parts by wt of monomer(s), 0.01-10 parts by wt of an emulsifier, 0.01-10 parts by wt of an initiator for polymerization and 50-90 parts by wt of water, wherein the components of said composition are mixed and stirred to be emulsified and then the resulting mixture is heated to 60-70° C. to be polymerized.

7. The method of manufacturing photochromic nanocapsule composition according to claim 6, wherein said initiator is used for polymerization by adding before or after said emulsification.

8. An optical recording medium, a photochromic window, a display element, a plastic mirror, a photochromic filter, a photosensing drum, a recording element, a solar battery, a lens, a fiber, a photochromic recording and image part, or a pharmaceutical drug which contain said photochromic nanocapsule composition according to claims 1-6.
